(19) United States
(12) Patent Application Publication
(10) Pub. No.: US 2018/0037653 A1
(43) Pub. Date: Feb. 8, 2018

(54) TREATMENT FOR CHRONIC LYMPHOCYTIC LEUKEMIA (CLL)

(71) Applicant: Xencor, Inc., Monrovia, CA (US)

(72) Inventors: Paul Foster, Monrovia, CA (US); John Byrd, Columbus, OH (US)

(21) Appl. No.: 15/704,734

(22) Filed: Sep. 14, 2017

Related U.S. Application Data
(62) Division of application No. 15/318,754, filed on Dec. 14, 2016.
(60) Provisional application No. 62/012,423, filed on Jun. 16, 2014.

(30) Foreign Application Priority Data
Jul. 4, 2014 (EP) 14175714.6

(51) Int. Cl. C07K 16/28; A61K 39/00

(52) U.S. Cl. CPC C07K 16/2803; C07K 2317/94; A61K 2039/505; A61K 2039/545; C07K 2317/565

(57) ABSTRACT

The present disclosure relates to the treatment of chronic lymphocytic leukemia. Monoclonal antibody XmAb5574 is efficacious when administered to patient at certain dosage regimens.

### CROSS REFERENCE

This application is a divisional of U.S. application Ser. No. 15/318,754 filed Dec. 14, 2016, which is the national phase of PCT/US2015/035722 filed Jun. 15, 2015; which claims the benefit of EP 14175714.6 filed Jul. 4, 2014 and U.S. provisional application Ser. No. 62/012,423 filed Jun. 16, 2014, each of which is incorporated by reference in its entirety.

### BACKGROUND OF THE INVENTION

Chronic Lymphocytic Leukemia (CLL) is a B-cell malignancy and the most prevalent form of adult leukemia. The disease is currently incurable outside of allogeneic stem cell transplantation. Patients diagnosed with or progressing to advanced disease have a mean survival of 18 months to 3 years. Unfortunately these patients with advanced disease are also more refractory to conventional therapy.

The have been advances in the treatment of CLL, such as the introduction of purine nucleoside analogs and the CD20 monoclonal antibody rituximab. However, with single agent rituximab treatment complete responses (CR) and extended remissions are rare. This can be improved upon by combining rituximab with traditional cytotoxic agents such as fludarabine or fludarabine and cyclophosphamide. Other CD20-specific antibodies, such as ofatumumab and obinutuzumab, are used or tested for the treatment of CLL as well. Other targets that have been evaluated include CD52. Alemtuzumab, an antibody specific for CD52, has shown efficacy in relapsed or treatment-naïve CLL, but its use is limited by significant infectious toxicity.

Another antibody target molecule for the treatment of CLL is CD19. CD19 is a 95-kDa transmembrane glycoprotein of the immunoglobulin superfamily containing two extracellular immunoglobulin-like domains and an extensive cytoplasmic tail. The protein is a pan-B lymphocyte surface receptor and is ubiquitously expressed from the earliest stages of pre-B cell development onwards until it is down-regulated during terminal differentiation into plasma cells. It is B-lymphocyte lineage specific and not expressed on hematopoietic stem cells and other immune cells, except some follicular dendritic cells. CD19 functions as a positive regulator of B cell receptor (BCR) signaling and is important for B cell activation and proliferation and in the development of humoral immune responses. It acts as a co-stimulatory molecule in conjunction with CD21 and CD81 and is critical for B cell responses to T-cell-dependent antigens. Upon ligand binding, the cytoplasmic tail of CD19 is physically associated with a family of tyrosine kinases that trigger downstream signaling pathways via the src-family of protein tyrosine kinases.

CD19 is an attractive target for cancers of lymphoid origin since it is highly expressed in nearly all chronic lymphocytic leukemia (CLL) and non-Hodgkin's lymphomas (NHL), as well as many other different types of leukemias, including acute lymphocytic leukemia (ALL) and hairy cell leukemia (HCL).

The clinical development of CD19 directed antibodies had previously been limited by the internalization of the CD19 antigen, however, improved antibody modification technology has restored this potential therapeutic target. XmAb5574 (aka MOR00208) is an Fc engineered humanized monoclonal antibody that binds CD19. XmAb5574 has been optimized using Xencor's proprietary XmAb® technology, which applies a novel method of humanization that maximizes the human sequence content, enhances affinity for antigen, and engineers the Fc region to increase binding affinity for various Fc gamma receptors (FcγR). In particular, binding to the human V158 polymorphic variant of FcγRIIIa has been increased 37 fold and binding to the human F158 polymorphic variant of FcγRIIIa has been increased by 137 fold relative to the non-engineered IgG1 analog of XmAb5574. The resulting antibody possesses variable modes of significantly increased tumor cytotoxicity relative to the murine mAb 4G7 or the non-engineered, chimeric 4G7 anti-CD19 antibody. The increase in binding of XmAb5574 Fc to FcγR, due to XmAb engineered mutations, significantly enhances in-vitro antibody dependent cell-mediated cytotoxicity (ADCC), antibody dependent cell-mediated phagocytosis (ADCP), and direct cytotoxic effects (apoptosis) on tumor relative to the unmodified antibody. XmAb5574 has not been shown to mediate complement dependent cytotoxicity.

### SUMMARY OF INVENTION

The present invention relates to the certain surprising findings observed in the first in human clinical trial with the Fc engineered CD19 monoclonal antibody XmAb5574 in patients with relapsed or refractory CLL.

A clinical trial studying XmAb5574 (aka MOR00208) in CLL has been completed. See Woyach et al. “2894 Final Results of a Phase I Study of the Fc Engineered CD19 Antibody XmAb5574 (MOR00208) in Patients with Relapsed or Refractory Chronic Lymphocytic Leukemia (CLL) or Small Lymphocytic Lymphoma (SLL)”, 54^{th} ASH Annual Meeting and Exposition, Dec. 9, 2012. There it was reported that responses occurred at the 6, 9 and 12 mg/kg dose levels.

In order to identify an appropriate dose for further study, a thorough evaluation of the pK data and clinical responses was completed. Surprisingly a statistically significant correlation was observed between Progression Free Survival of patients and a dosing of 9 mg/kg or more.

Accordingly, appropriate dose selection for patients can be selected based upon such findings.

### FIGURE LEGENDS

FIG. 1 shows the best percent change in lymphocyte count from baseline of the patients of the present study. Blood disease cleared in most patients, with a median reduction in absolute lymphocyte count from baseline of 90.8%.

FIGS. 2A and 2B. show the best lymph node reduction for all patients. Changes are shown as the sum of product diameters of lymph nodes by physical exam (FIG. 2A) or as assessed by CT (FIG. 2B).

FIGS. 3A-3C shows the Progression Free Survival for all patients (FIG. 3A), those who received up to 9 doses on all dose levels (FIG. 3B), and those who were included in the extended-dosing cohort (FIG. 3C).

FIG. 4 shows the amino acid sequence of the variable domains of antibody XmAb5574.

FIG. 5 shows the amino acid sequence of the heavy and light chain Fc regions of XmAb5574.

FIG. 6 shows a comparison between the Progression Free Survival of patients receiving a dose of less than 9 mg/kg as compared to patients receiving a dose of 9 mg/kg or more.

### CERTAIN EMBODIMENTS OF THE INVENTION

In certain embodiments the present disclosure relates to an antibody specific for CD19 wherein said antibody cross-competes with an antibody comprising an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6) for use in the treatment of chronic lymphocytic leukemia, wherein said antibody is administered at a dose of 9 mg/kg or more.

In certain embodiments the present disclosure relates to a method of treating chronic lymphocytic leukemia comprising administering an antibody specific for CD19 wherein said antibody cross-competes with an antibody comprising an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6), wherein said antibody is administered at a dose of 9 mg/kg or more.

In certain embodiments said antibody comprises an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6).

In certain embodiments said antibody comprises a variable heavy chain of the sequence EVQLVESGGGLVKPGGSLKLSCAASGYTFTSYVMHWVRQAPGKGLEWIGYINPYNDGT KYNEKFQGRVTISSDKSISTAYMELSSLRSEDTAMYYCARGTYYYGTRVFDYWGQGTLV TVSS (SEQ ID NO: 7) and a variable light chain of the sequence DIVMTQSPATLSLSPGERATLSCRSSKSLQNVNGNTYLYWFQQKPGQSPQLLIYRMSNL NSGVPDRFSGSGSGTEFTLTISSLEPEDFAVYYCMQHLEYPITFGAGTKLEIK (SEQ ID NO: 8).

In certain embodiments said antibody comprises a heavy chain constant domain of the sequence ASTKGPSVFPLAPSSKSTSGGTAALGCLVKDYFPEPVTVSWNSGALTSGVHTFP AVLQSSGLYSLSSWTVPSSSLGTQTYICNVNHKPSNTKVDKKVEPKSCDKTHTCPPCP APELLGGPDVFLFPPKPKDTLMISRTPEVTCVVVDVSHEDPEVQFNWYVDGVEVHNAKT KPREEQFNSTFRWSVLTWHQDWLNGKEYKCKVSNKALPAPEEKTISKTKGQPREPQ VYTLPPSREEMTKNQVSLTCLVKGFYPSDIAVEWESNGQPENNYKTTPPMLDSDGSFFL YSKLTVDKSRWQQGNVFSCSVMHEALHNHYTQKSLSLSPGK (SEQ ID NO: 9).

In certain embodiments said antibody is administered at a level that achieves a total exposure to said patent measured by area under the curve (AUC) of 14,500 μg*day/mL or more.

In certain embodiments said antibody is administered at least once weekly over at least eight weeks.

In certain embodiments said antibody is administered intravenously or subcutaneously.

### DETAILED DESCRIPTION OF THE INVENTION

The term “antibody” means monoclonal antibodies, including any isotype, such as, IgG, IgM, IgA, IgD and IgE. An IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are referred to as CDR1, CDR2, and CDR3, numbered sequentially from the N-terminus. The more highly conserved portions of the variable regions outside of the CDRs are called the “framework regions”. An “antibody fragment” means an Fv, scFv, dsFv, Fab, Fab′ F(ab′)2 fragment, or other fragment, which contains at least one variable heavy or variable light chain, each containing CDRs and framework regions.

“VH” refers to the variable region of an immunoglobulin heavy chain of an antibody, or antibody fragment. “VL” refers to the variable region of the immunoglobulin light chain of an antibody, or antibody fragment.

The “CDRs” herein are defined by either Chothia et al or Kabat et al. See Chothia C, Lesk A M. (1987) Canonical structures for the hypervariable regions of immunoglobulins. J Mol Biol., 196(4):901-17, which is incorporated by reference in its entirety. See Kabat E. A, Wu T. T., Perry H. M., Gottesman K. S. and Foeller C. (1991). Sequences of Proteins of Immunological Interest. 5th edit., NIH Publication no. 91-3242, US Dept. of Health and Human Services, Washington, D.C.

The term “CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12.

Human CD19 has the amino acid sequence of:

(SEQ ID NO: 10)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR.

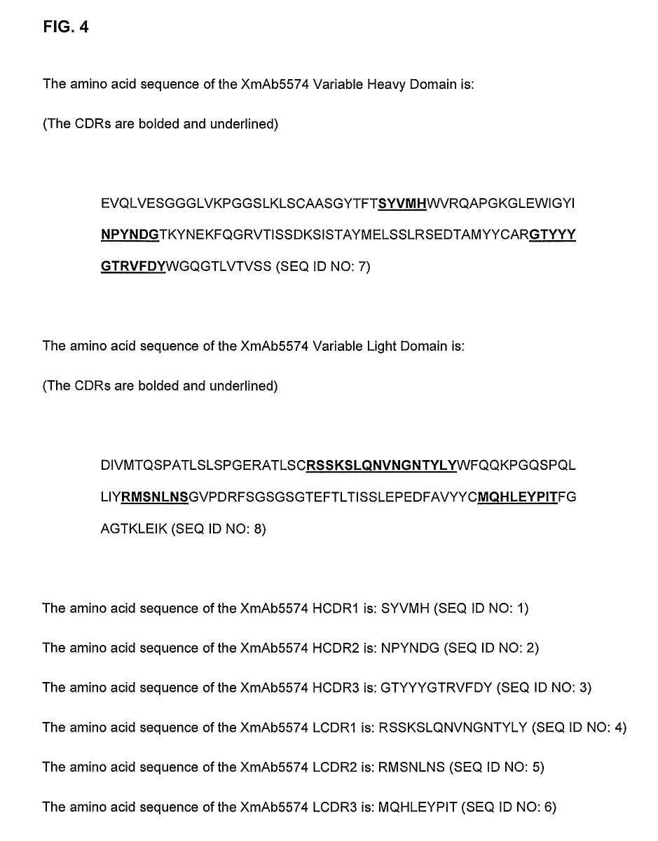

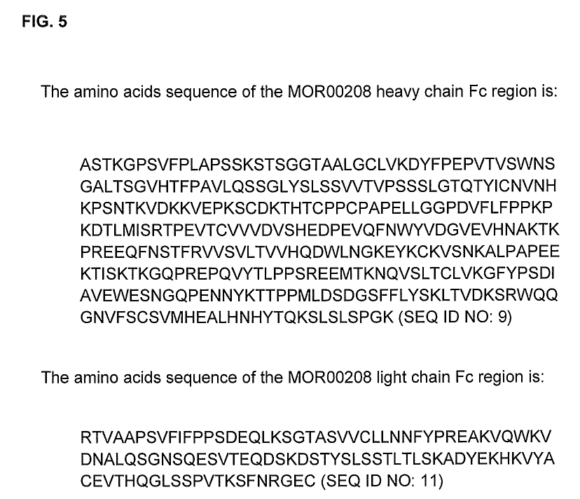

“MOR00208” is an anti-CD19 antibody. The amino acid sequence of the variable domains is provided in FIG. 4. The amino acid sequence of the heavy and light chain Fc regions of MOR00208 is provided in FIG. 5. “MOR208,” “MOR00208” and “XmAb5574” are used as synonyms to describe the antibody shown in FIGS. 4 and 5. The MOR00208 antibody is described in U.S. patent application Ser. No. 12/377,251, which is incorporated by reference in its entirety. The CDR regions of XmAb5574 are as follows:

HCDR1 sequence:
(SEQ ID NO: 1)
SYVMH,
HCDR2 sequence:
(SEQ ID NO: 2)
NPYNDG,
HCDR3 sequence:
(SEQ ID NO: 3)
GTYYYGTRVFDY,
LCDR1 sequence:
(SEQ ID NO: 4)
RSSKSLQNVNGNTYLY,
LCDR2 sequence:
(SEQ ID NO: 5)
RMSNLNS,
and
LCDR3 sequence:
(SEQ ID NO: 6)
MQHLEYPIT.

A pharmaceutical composition includes an active agent, e.g. an antibody for therapeutic use in humans. A pharmaceutical composition may additionally include pharmaceutically acceptable carriers or excipients.

“Administered” or “administration” refers to the delivery of a pharmaceutical composition by an injectable form, such as, for example, an intravenous, intramuscular, intradermal or subcutaneous route or mucosal route, for example, as a nasal spray or aerosol for inhalation or as an ingestable solution, capsule or tablet.

The antibody which is administered according to the present disclosure is administered to the patient in a therapeutically effective amount. A “therapeutically effective amount” refers to an amount sufficient to cure, alleviate or partially arrest the clinical manifestations of a given disease or disorder, i.e. CLL, and its complications. The present disclosure surprisingly found that XmAb5574 is able to treat CCL at a dose of as low as 9 mg/kg (mg antibody per kilogram body weight). Therefore, in certain embodiments the antibodies of the present disclosure are administered at 9 mg/kg. In alternative embodiments the antibodies of the present disclosure are administered at 12 mg/kg. In yet other embodiments the antibodies of the present disclosure are administered at 15 mg/kg or more.

“Cmax” refers to the highest plasma concentration of the antibody observed within the sampling interval.

“AUC” or “area under the curve” refers to the area under the plasma or serum concentration-time curve of the molecule analyzed (e.g. the drug), as calculated by the trapezoidal rule over the complete sample collection interval.

The drug dose that leads to a therapeutically effect can also be described in terms of the total exposure to a patient measured by area under the curve.

In certain embodiments the antibodies of the present disclosure the antibody is administered at a level that achieves a total exposure to said patent measured by area under the curve (AUC) of 14,500 μg*day/mL or more. In alternative embodiments the antibodies of the present disclosure the antibody is administered at a level that achieves a total exposure to said patent measured by area under the curve (AUC) of 17,500 μg*day/mL or more.

The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or clinical scientist.

The antibody of the present disclosure can be administered at different time points and the treatment cycle may have a different length. The antibodies may be administered daily, every other day, three times a week, weekly or biweekly. The antibodies may also be administered over at least four weeks, over at least five weeks, over at least six weeks, over at least seven weeks, over at least eight weeks, over at least nine weeks, over at least ten weeks, over at least eleven weeks or over at least twelve weeks. In certain embodiments of the present disclosure the antibody is administered at least once weekly over at least eight weeks.

“Administered” or “administration” includes but is not limited to delivery by an injectable form, such as, for example, an intravenous, intramuscular, intradermal or subcutaneous route or mucosal route, for example, as a nasal spray or aerosol for inhalation or as an ingestable solution, capsule or tablet. In certain embodiments the antibody is administered intravenously. In other embodiments the antibody is administered subcutaneously.

“Chronic lymphocytic leukemia” or “CLL” as used herein, refers to a cancer of the lymphocyte lines of white blood cells and includes the Rai and Binnet classification subtypes subcategories. Disease progression in “chronic” lymphocytic leukemia is gradual in comparison to “acute” lymphocytic leukemia. CLL is the most frequent form of leukemia in adults accounting for 25% of all leukemias (approximately 10,000 new CLL cases yearly in the United States (US)). SLL (small lymphocytic lymphoma) is a certain form of CLL, which presents primarily in the lymph nodes. CLL and SLL are considered the same underlying disease, just with different appearances. In certain embodiments of the present disclosure said CLL is relapsed CLL. In other embodiments of the present disclosure said CLL is refractory CLL.

“Cross competes” means the ability of an antibody or another binding agent to interfere with the binding of other antibodies or binding agents to CD19 in a standard competitive binding assay. The ability or extent to which an antibody or other binding agent is able to interfere with the binding of another antibody or binding molecule to CD19, and, therefore whether it can be said to cross-compete according to the invention, can be determined using standard competition binding assays. One suitable assay involves the use of the Biacore technology (e.g. by using the BIAcore 3000 instrument (Biacore, Uppsala, Sweden)), which can measure the extent of interactions using surface plasmon resonance technology. Another assay for measuring cross-competing uses an ELISA-based approach. A high throughput process for “epitope binning” antibodies based upon their cross-competition is described in International Patent Application No. WO 2003/48731.

The term “epitope” includes any protein determinant capable of specific binding to an antibody or otherwise interacting with a molecule. Epitopic determinants generally consist of chemically active surface groupings of molecules such as amino acids or carbohydrate or sugar side chains and can have specific three-dimensional structural characteristics, as well as specific charge characteristics. An epitope may be “linear” or “conformational.” The term “linear epitope” refers to an epitope with all of the points of interaction between the protein and the interacting molecule (such as an antibody) occur linearally along the primary amino acid sequence of the protein (continuous). The term “conformational epitope” refers to an epitope in which discontinuous amino acids that come together in three dimensional conformation. In a conformational epitope, the points of interaction occur across amino acid residues on the protein that are separated from one another.

“Binds the same epitope as” means the ability of an antibody or other binding agent to bind to CD19 and having the same epitope as the exemplified antibody. The epitopes of the exemplified antibody and other antibodies to CD19 can be determined using standard epitope mapping techniques. Epitope mapping techniques, well known in the art include Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al, (1984) Proc. Natl. Acad. Sci. USA 8:3998-4002; Geysen et al, (1985) Proc. Natl. Acad. Sci. USA 82:78-182; Geysen et al, (1986) Mol. Immunol. 23:709-715. Similarly, conformational epitopes are readily identified by determining spatial conformation of amino acids such as by, e.g., hydrogen/deuterium exchange, x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al, (1981) Proc. Natl. Acad. Sci USA 78:3824-3828; for determining antigenicity profiles, and the Kyte-Doolittle technique, Kyte et al, (1982) J. Mol. Biol. 157: 105-132; for hydropathy plots.

### EXAMPLES

### Example 1: Patient Selection

The study is multicenter, open-label, single arm phase I dose escalation study. Patients were eligible to participate in the study if they were >18 years of age, met the diagnostic criteria for CLL or SLL according to IWCLL 2008 guidelines (Hallek et al, Blood (2008) 111, 5446-545), had active disease requiring therapy, and had relapsed or refractory disease following at least one purine analog-containing regimen (or alternate regimen if there was a relative contraindication to purine analog therapy). Patients were required to have adequate kidney and liver function. Platelet count could not be <50,000/mm³ and absolute neutrophil count (ANC) was required to be ≧1,000/mm³ if white blood cell count (WBC) was <50,000/mm³. There was no limit for ANC in patients with WBC ≧50,000/mm³. Patients previously treated with alternate CD19 antibody therapeutics were excluded.

After providing written informed consent, 27 patients with relapsed or refractory CLL/SLL were enrolled to this Institutional Review Board-approved study conducted in accordance with the principles of the Declaration of Helsinki between Nov. 30, 2010 and Apr. 17, 2012. Each of these patients received at least 1 dose of therapy. Patient demographics are outlined in Table 1. The patients were generally high risk, with 14 (52%) having high-risk disease by Rai stage and 24 (89%) having IGHV unmutated disease. On FISH analysis, 8 (30%) had del(11q22.3) and 10 (37%) had del(17p13.1). Patients had a median of 4 prior therapies, with a range of 1-13.

TABLE 1
Demographics of Patients Treated in the present Study

| Characteristic | Number |
|---|---|
| Total Patients | 27 |
| Median Age | 66 (range 40-84) |
| Gender | |
| Male | 18 (67%) |
| Female | 9 (33%) |
| ECOG Performance Status | |
| 0 | 11 (41%) |
| 1 | 15 (56%) |
| 2 | 1 (4%) |
| Median number of prior | 4 (range 1-13) |
| therapies | |
| Prior CD20 antibody | 27 (100%) |
| IgVH unmutated | 24 (89%) |
| Cytogenetics | |
| Del(13q14.3) | 16 (59%) |
| Trisomy 12 | 4 (15%) |
| Del(11q22.3) | 8 (30%) |
| Del(17p13.1) | 10 (37%) |
| Median B2M | 3.6 (range 1.6-9.3 mg/L) |
| Splenomegaly | 16 (59%) |

### Example 2: Study Design

Patients were enrolled to this dose escalation study initially in an accelerated manner to limit the number of patients potentially exposed to a sub-therapeutic dose. During the accelerated dose escalation, one patient was enrolled to each cohort and dose escalation could occur after that patient completed cycle 1 if there was no dose limiting toxicity (DLT) or grade 2 treatment-related toxicities. If a DLT or grade 2 toxicity was reached, or beginning at the 3 mg/kg dose level, the dose escalation strategy would revert to standard 3×3 design. In this design, 3 patients are initially enrolled to each cohort, and if 0 patients have a DLT, escalation would occur. If 1 patient experiences a DLT, expansion to 6 patients would occur, and if no patient experiences a DLT, dose escalation would occur. If 2 patients in a cohort experience a DLT, the next lower dose would be expanded and considered as the MTD or recommended phase 2 dose.

Patients received 9 total infusions of XmAb5574: days 1, 4, 8, 15, and 22 of cycle 1, and days 1, 8, 15, and 22 of cycle 2. Once the first 5 patients had been treated in the maximal planned cohort, additional patients enrolled at this dose level who had at least stable disease after 2 cycles were given the option of continuing to receive XmAb5574 every 28 days for an additional 4 infusions.

### Example 3: Correlative Laboratory Studies

All patients enrolled to this study had stimulated cytogenetics, FISH, and IGHV mutational status performed at baseline as previously described (Byrd et al, J Clin Oncol (2006) 24, 437-43; Woyach et al, Br J Haematol (2010) 148, 754-9. Flow cytometry was performed at baseline and designated time points. After viability assessment, samples were stained using PrepPlus2 automated staining system (Beckman Coulter) using five color whole blood staining technique with panels of directly conjugated monoclonal antibodies. Following 30 minutes of incubation at room temperature in the dark the red cells were lysed using TQ-prep instrument and ImmunoPrep reagent (both from Beckman Coulter) according to manufacturer's recommendations. Samples were analyzed on FC500 flow cytometer (Beckman-Coulter) equipped with CXP software version 2.1 (Beckman Coulter). Multiparametric analysis was performed with gating strategy based on CD45 staining and light side scatter characteristics that allow good separation of lymphocyte, monocyte and myeloid cell populations. Detailed immunophenotypic characterization of the lymphocyte gate was performed using Prism plot algorithm (Beckman Coulter). Enumeration of B cells was performed in the context of CD24 antigen expression as an alternative pan B cell marker in CLL, as administration of XmAb5574 renders CD19 undetectable. Evaluation of CLL B cells was therefore based on enumeration of CD24 positive B cells that expressed other CLL antigens such as CD5, CD43 and CD79b.

Serum samples were assayed for XmAb5574 by Prevalere Life Sciences, a division of ICON Development Solutions, LLC (Whitesboro, N.Y., USA) using a validated method. Prevalere executed PK testing using a validated ELISA method for quantitation of XmAb5574 in human serum. The lower limit of detection was 0.2 ng/mL.

Pharmacokinetic parameters including maximum concentration (Cmax), time of Cmax (Tmax), terminal phase half-life (t1/2), area under the serum concentration-time curve from time zero to infinity (AUC∞), clearance (CL), and volume of distribution (V) were estimated using either non-compartmental or compartmental methods, whichever best described the observed data. All PK parameters were computed using actual elapsed time to PK sampling event and to dose event start and stop, calculated relative to the first dose start of infusion. Dose used to compute PK parameters was the actual dose delivered during the infusion duration. Dose proportionality across dose levels was characterized by plotting Cmax and AUC∞ versus dose. Similarly, the kinetic parameters terminal half-life, Tmax, CL, and V across dose levels was to be characterized by plots of these parameters versus dose. Pharmacokinetic parameters were derived by fitting a two-compartment IV infusion model to the time concentration profiles for each patient using PK model 10 in the software WinNonlin Phoenix.

Analysis of serum human anti-human antibody (HAHA) was performed by Prevalere Life Sciences. Antibodies against XmAb5574 were measured in human serum using an electrochemiluminescent immunoassay method utilizing MSD technology with Ruthenylated (Sulfo-tagged) XmAb5574 and Biotinylated XmAb5574. The signal produced is proportional to the amount of anti-XmAb5574 antibody present. Study samples with a response at or above the assay cut point were considered potentially positive. Study samples with a response below the assay cut point were considered negative.

### Example 4: Toxicity and Response Assessment

Safety assessments were performed weekly while the patients were receiving therapy, and then every 4 weeks for an additional twelve weeks. Hematologic toxicity was graded according to the IWCLL 2008 criteria¹⁷ and non-hematologic toxicity was graded by the National Cancer Center Institute Common Terminology Criteria for Adverse Events version 4.0.

Responses were determined according to IWCLL 2008 guidelines (Hallek et al, Blood (2008) 111, 5446-545) which incorporate physical examination and clinical laboratory data as well as CT scan data for CLL, and per the 2007 IWG response criteria for SLL (Cheson et al, J Clin Oncol (2007) 25, 579-86. Responses were evaluated on Cycle 2 day 1, end of cycle 2, and 4, 8, and 12 weeks after the end of cycle 2. Progression free survival was measured from cycle 1 day 1 to the first date that recurrent or progressive disease or death due to any cause was documented. Patients were censored at the last follow-up date if they were lost to follow up or chose not to provide future information.

### Example 5: Treatment Administered

One patient was accrued each to the 0.3 mg/kg and 1 mg/kg dose cohort. Three patients each were accrued to the 3 mg/kg, 6 mg/kg, and 9 mg/kg dose cohorts. 16 patients, inclusive of an expansion cohort, were accrued to the maximum dose evaluated, 12 mg/kg. All 27 patients enrolled received at least 1 dose of XmAb5574, with 22 patients receiving all 9 of the initial planned doses of therapy. Of the 5 patients who did not receive all 9 doses, 2 experienced disease progression, 1 experienced unacceptable adverse events (DLT of grade 4 neutropenia), 1 was removed from study by the treating physician and 1 completed the study but missed one dose due to an adverse event (grade 3 thrombocytopenia). No patients had dose reductions during the trial. 5 patients had at least 1 dose delayed for an adverse event. 18 patients had the infusion paused at least once for infusion reactions.

Eight patients participated in the maintenance cohort to assess the safety of this for future investigation. One patient received only 3 additional infusions, and the other 7 received all 4 planned additional infusions.

### Example 6: Adverse Events

XmAb5574 was generally well tolerated, with only 1 patient discontinuing therapy due to toxicity. All treatment-related adverse events are outlined in Table 2. One case of dose limiting toxicity (DLT) of grade 4 neutropenia (lasting >7 days) was seen at the 12 mg/kg dose. 5 patients experienced grade 3 or 4 treatment-related adverse events, which included neutropenia (3 patients), thrombocytopenia (2 patients), increased aspartate aminotransferase (AST; 1 patient), febrile neutropenia (1 patient), and tumor lysis syndrome (1 patient).

TABLE 2
Adverse Events at Least Possibly Attributable to XmAb5574

| | 0.3 mg/kg | 1 mg/kg | 3 mg/kg | 6 mg/kg | 9 mg/kg | 12 mg/kg | Total | |
|---|---|---|---|---|---|---|---|---|
| Toxicity | (N = 1) | (N = 1) | (N = 3) | (N = 3) | (N = 3) | (N = 16) | (%) | |
| Any Event | 1 | 1 | 3 | 3 | 2 | 14 | 24 | (88.9) |
| Dose Limiting | | | | | | | | |
| Toxicities | | | | | | | | |
| Grade 4 Neutropenia | | | | | | 1 | 1 | (3.7) |
| lasting ≧7 days with | | | | | | | | |
| febrile neutropenia | | | | | | | | |
| Other Grade ¾ | | | | | | | | |
| Toxicities | | | | | | | | |
| Neutropenia | | 1 | | | | 1 | 2 | (7.4) |
| Thrombocytopenia | | | | | | 2 | 2 | (7.4) |
| Tumor Lysis | | | | | | 1 | 1 | (3.7) |
| Syndrome | | | | | | | | |
| Increased AST | | | | | | 1 | 1 | (3.7) |
| Grade ½ Toxicities | | | | | | | | |
| Occurring in >1 | | | | | | | | |
| Patient | | | | | | | | |
| Infusion Reaction | 1 | 1 | 1 | 2 | 2 | 11 | 18 | (66.7) |
| Increased AST | 1 | | 2 | 1 | | | 4 | (14.8) |
| Increased ALT | | | 2 | 1 | | 2 | 5 | (18.5) |
| Neutropenia | 1 | | | | | 1 | 2 | (7.4) |
| Thrombocytopenia | | | | | 1 | 2 | 3 | (11.1) |
| Fever | 1 | | 1 | | 1 | 1 | 4 | (14.8) |
| Chills | 1 | | 1 | 1 | | | 3 | (11.1) |
| Peripheral Sensory | | | | 1 | | 2 | 3 | (11.1) |
| Neuropathy | | | | | | | | |
| Diarrhea | | | | 1 | | 1 | 2 | (7.4) |
| Flushing | 1 | | | | | 1 | 2 | (7.4) |
| Hyperuricemia | | | 1 | | | 1 | 2 | (7.4) |
| Hypocalcemia | | | 1 | | | 1 | 2 | (7.4) |
| Increased Lipase | | | 1 | | | 1 | 2 | (7.4) |

Grade 1 and 2 toxicities assessed as possibly related to XmAb5574 that occurred in more than 10% of patients included infusion reactions, increased aspartate aminotransferase (AST), increased alanine aminotransferase (ALT), neutropenia, thrombocytopenia, fever, chills, and peripheral neuropathy. Infusion reactions were the most common toxicity which occurred in 67% of patients, however, no grade 3 or 4 infusion reactions were seen. In general, this reaction occurred early in the infusion, with the majority occurring within the first 15 minutes, and quickly responded to slowing of infusion rate or pause of dose. All infusion reactions occurred during the first infusion and responded to treatment. II patients completed the Day 1 infusion and only one patient had recurrence of infusion symptoms during Day 1. No infusion reactions occurred during subsequent infusions for any of the patients.

In summary, no MTD (maximum tolerated dose) was reached in the dose levels examined, and the drug was well tolerated. While infusion reactions were common, these were manageable in all cases with supportive care, and generally did not recur during subsequent doses. Grade 3 and 4 toxicities were primarily hematologic and in most cases did not require discontinuation of therapy. Infectious toxicities were notably low, with febrile neutropenia occurring in only one patient. One patient did develop TLS (tumor lysis syndrome) which required rasburicase and intravenous fluids, however, subsequent infusions were tolerated without incident. It is notable that this patient had received no prior chemotherapy prior to receipt of XmAb5574. Therefore, the adverse events observed are manageable and encouraging for the further clinical development of XmAb5574.

### Example 7: HAHA Analysis

8 total patients out of 27 evaluated tested positive for HAHA antibodies. However, all of these patients had positive HAHA at pretreatment, and no patient had HAHA titer increase from pre-dose level. Therefore, and very importantly, no evidence of immunogenicity was observed.

### Example 8: Response to Therapy

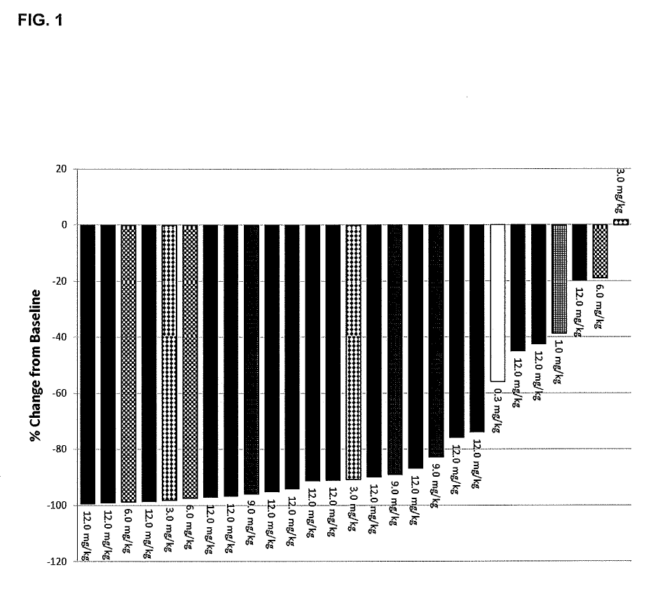

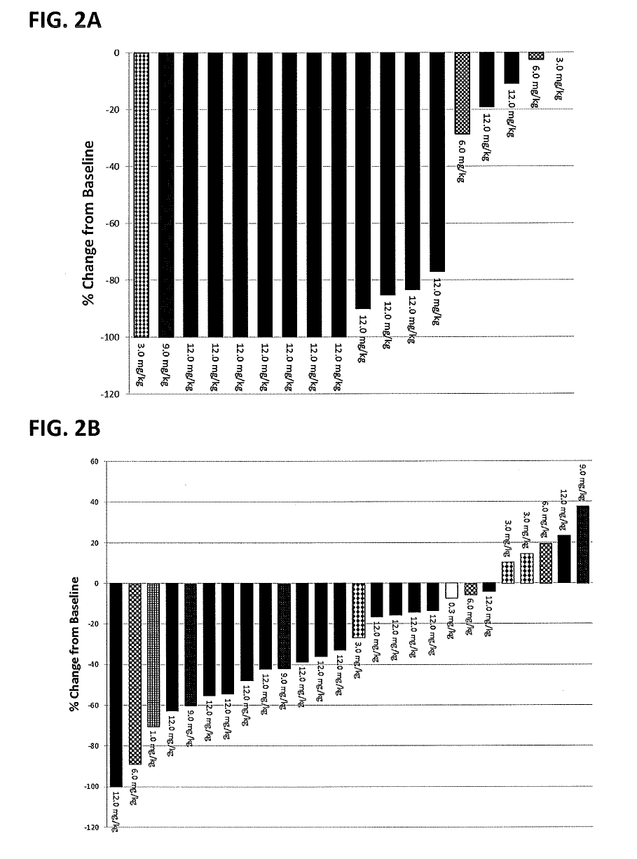

27 patients were evaluable for response. Blood disease cleared in most patients, with a median reduction in absolute lymphocyte count from baseline of 90.8% (FIG. 1). On the basis of physical exam and laboratory studies alone, 18 patients (66.7%) achieved a partial response (PR), and the remaining 9 patients (33.3%) achieved stable disease (SD). Best lymph node reduction for all patients is seen in FIG. 2. Using CT criteria as well as exam and laboratory data, 8 patients (29.6%) achieved a PR with an additional 16 patients (59.3%) achieving SD. Two patients had progressive disease by CT criteria. No patient dosed below 3 mg/kg had an objective response. Evaluating only the 16 patients at the 12 mg/kg dose level, which is the recommended phase II dose, 12 patients (75%) had a PR by physical exam criteria and 6 patients (37.5%) had a PR by CT criteria. Patients who responded by physical exam criteria tended to do so quickly, with 14/18 patients achieving a PR at the first response evaluation (cycle 2 day 1). CT responses lagged somewhat, with 3/8 patients achieving a PR at cycle 2 day 28, 3 at the 4 week follow-up time point, and 2 patients in the expansion cohort achieving a PR at cycle 5 and cycle 7, respectively.

When looking at baseline characteristics related to response by physical exam or CT criteria, baseline lymph node size did appear to be associated with response, where patients with the largest lymph node ≦5 cm (n=18) had a 77.8% PR rate by exam criteria and 38.9% by CT criteria, where those patient with largest lymph node >5 cm (n=9) had a 44.4% PR rate by exam and 11.1% by CT. Cytogenetic abnormalities by FISH, including del(17p13.1) did not appear to be associated with response, with 60% of patients with del(17p13.1) (6 of 10 patients) achieving a PR by exam criteria and 30% achieving a PR by CT criteria.

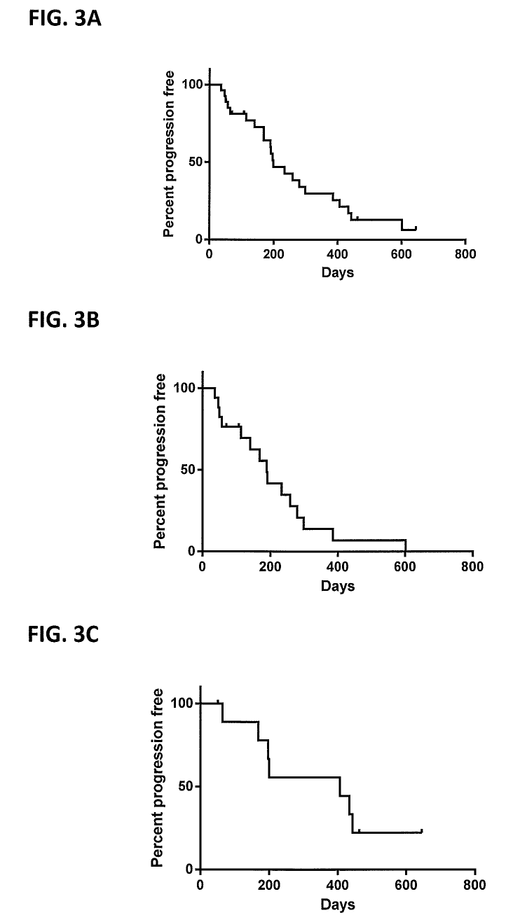

At evaluation 12 weeks post cycle 2 day 28 of XmAb5574, 5 patients (18.5%) had progressed by CT criteria, and 8 (29.6%) by physical exam criteria. No patient died during therapy or during this 12 week observation period. Progression Free Survival (PFS) was defined from the time of first dose to the time of progression or death, whichever came first. PFS for all patients, including those in extended treatment cohort, was 199 days (FIG. 3A; 95% Cl: 168-299 days). For all patients on all dose levels who received 9 doses or less, PFS was 189 days (FIG. 3B), and for patients on the extended treatment cohort alone, PFS was 420 days (FIG. 3C; 95% Cl: 168 days-not evaluable).

Since the Phase I clinical trial was not designed to demonstrate any conclusive signs of efficacy, the results observed with certain concentrations of XmAb5574 are highly encouraging and surprising. 67% or patients achieved a PR by clinical criteria when treated with XmAb5574 as a single agent antibody. These responses compare favorably with results for rituximab given on a weekly schedule (Huhn et al, Blood (2001) 98, 1326-31; Itala et al, Eur J Haematol (2002) 69, 129-34) as well as ofatumumab (Wierda et al, J Clin Oncol (2010) 28, 1749-55.

Efficacy observed was particularly pronounced in patients receiving a dose of 9 mg/kg or more. This is reflected in a reduction in absolute lymphocyte count (see FIG. 1) and in lymph node reduction (sees FIG. 2). Responses to XmAb5574 were also fairly durable. Response duration was prolonged in those patients receiving maintenance therapy. Progression Free Survival is the length of time during and after the treatment of a disease that a patient lives with the disease but it does not get worse. This is an important endpoint of a clinical trial, and an indicator of effectiveness in patients.

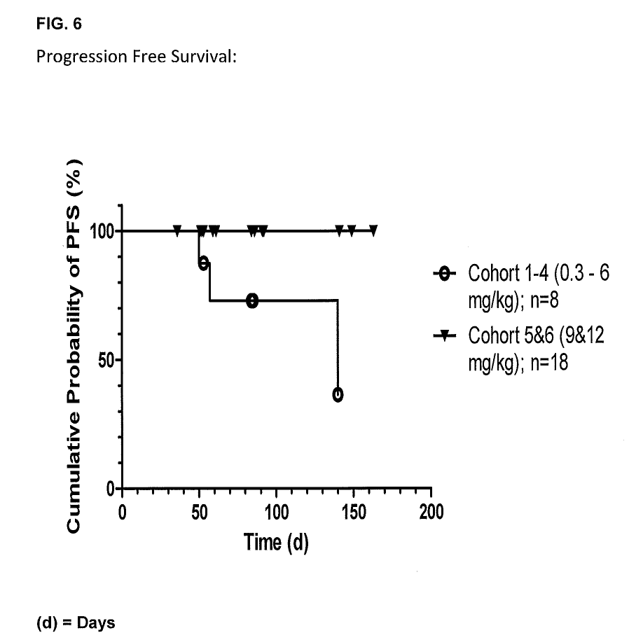

Progression Free Survival is shown in FIG. 6. FIG. 6 is Kaplan-Meier plot of Progression Free Survival (PFS) based on computed tomography (CT). The symbols highlight censored events only. For calculation of PFS, only dosing cycles 1 and 2 were considered. One Patient in Cohort 6 was excluded from data analysis since the patient received only 2 of 9 infusions and left the clinical study on Day 8.

Statistical analysis of the data shown in FIG. 6 is shown in Table 3.

TABLE 3
Log-rank (Mantel-Cox) Test

| | |
|---|---|
| Chi square | 5.574 |
| df | 1 |
| P value | 0.0182 |
| P value summary | * |
| Are the survival curves significantly different? | Yes |

Gehan-Breslow-Wilcoxon Test

| | |
|---|---|
| Chi square | 4.785 |
| df | 1 |
| P value | 0.0287 |
| P value summary | * |
| Are the survival curves significantly different? | Yes |

Statistical testing was performed with GraphPad Prism, V5.02

The patients shown in FIG. 6 were dosed at either 0.3, 1, 3, 6, 9, or 12 mg/kg. Previously it has been reported that responses occurred at 6, 9 and 12 mg/kg.

Surprisingly, patients dosed with 9 mg/kg or more showed a statistically significantly increase in progression free survival (PFS) as compared to patients receiving lower doses, such as 6 mg/kg. This is surprising and could not have been predicted based upon the existence of responses at dose levels of 6, 9 and 12 mg/kg.

Accordingly, it can be expected that doses of 9 mg/kg or more yield better clinical effectiveness as compared to lower doses. This is further supported by a review of the clinical responses in Table 4.

TABLE 4

| XmAb5574 Dose Cohort (mg/kg) | | | | | | |
|---|---|---|---|---|---|---|
| 0.3 | 1.0 | 3.0 | 6.0 | 9.0 | 12.0 | Total |
| (N = 1) | (N = 1) | (N = 3) | (N = 3) | (N = 3) | (N = 16) | (N = 27) |

IWCLL 2008 Guideline

| | | | | | | | |
|---|---|---|---|---|---|---|---|
| CR | 0 | 0 | 0 | 0 | 0 | 0 | 0 |
| PR | 0 | 0 | 0 | 1 (33.3%) | 1 (33.3%) | 6 (37.5%) | 8 (29.6%) |
| SD | 1 (100.0%) | 1 (100.0%) | 2 (66.7%) | 1 (33.3%) | 2 (66.7%) | 9 (56.3%) | 16 (59.3%) |
| PD | 0 | 0 | 1 (33.3%) | 1 (33.3%) | 0 | 0 | 2 (7.4%) |
| Unknown | 0 | 0 | 0 | 0 | 0 | 1 (6.3%) | 1 (3.7%) |

NCI-WG CLL 1996 Guideline

| | | | | | | | |
|---|---|---|---|---|---|---|---|
| CR | 0 | 0 | 0 | 0 | 0 | 0 | 0 |
| PR | 0 | 0 | 2 (66.7%) | 1 (33.3%) | 3 (100.0%) | 12 (75.0%) | 18 (66.7%) |
| SD | 1 (100.0%) | 1 (100.0%) | 1 (33.3%) | 2 (66.7%) | 0 | 4 (25.0%) | 9 (33.3%) |
| PD | 0 | 0 | 0 | 0 | 0 | 0 | 0 |

In Table 4 it is shown that using the IWCLL 2008 Guidelines that patients treated with 9 mg/kg or more of MOR00208 did not show progressive disease. Whereas some patients treated with lower doses observed progressive disease.

### Example 9: Pharmacokinetic Analysis

Of the 27 patients, PK parameters for 25 of these patients fit a 2-compartment model. Neither the patient enrolled at 0.3 or 1 mg/kg fit the expected PK model, and all PK data presented will be from the 3 mg/kg cohort and above. Key PK parameters as evaluated assuming a single dose of MOR00208 only are summarized by cohort in Table 3. Clearance and volume of distribution are noted to be similar to other full length monoclonal antibodies, and distribution was limited to the systemic circulation as evidenced by an estimate of volume of distribution. Cₘₐₓ increased in a slightly less than dose-proportional manner, and AUC increased in a dose-proportional manner. Clearance and half-life showed no dose dependence. A trend of accumulation in concentration was observed with each infusion, and serum concentration of XmAb5574 reached a plateau suggestive of steady-state at or before infusion 9. Across the dose range from 3-12 mg/kg, half-life averaged 13.5 days, supporting dosing intervals of 1-3 weeks. Table 5: Key Pharmacokinetic Parameters assuming a single dose of MOR00208 only

TABLE 5

| | | | | | | | | Alpha | Beta | K10 | |
|---|---|---|---|---|---|---|---|---|---|---|---|
| | | Cmax | AUC∞ | CL | V1 | V2 | Vss | Half-Life | Half-Life | Half-Life | MRT |
| Cohort | | ng/mL | day*ng/mL | mL/day/kg | mL/kg | mL/kg | mL/kg | day | day | day | day |
| Cohort 3 | N | 3 | 3 | 3 | 3 | 3 | 3 | 3 | 3 | 3 | 3 |
| (3.0 mg/kg) | Mean | 43,309 | 465,218 | 6.542 | 67.25 | 67.18 | 134.4 | 0.6514 | 15.00 | 7.266 | 20.19 |
| | SD | 2,978 | 70,169 | 0.9330 | 3.552 | 46.10 | 42.55 | 0.4147 | 3.637 | 1.498 | 4.028 |
| | CV % | 6.9 | 15.1 | 14.3 | 5.3 | 68.6 | 31.7 | 63.7 | 24.2 | 20.6 | 20.0 |
| Cohort 4 | N | 3 | 3 | 3 | 3 | 3 | 3 | 3 | 3 | 3 | 3 |
| (6.0 mg/kg) | Mean | 102,363 | 880,104 | 6.99 | 56.45 | 30.74 | 87.19 | 0.7944 | 9.496 | 5.791 | 12.95 |
| | SD | 9,668 | 173,357 | 1.323 | 7.714 | 7.721 | 8.557 | 0.6252 | 3.169 | 1.595 | 3.835 |
| | CV % | 9.4 | 19.7 | 18.9 | 13.7 | 25.1 | 9.8 | 78.7 | 33.4 | 27.5 | 29.6 |
| Cohort 5 | N | 3 | 3 | 3 | 3 | 3 | 3 | 3 | 3 | 3 | 3 |
| (9.0 mg/kg) | Mean | 132,687 | 1,462,480 | 6.758 | 68.80 | 31.57 | 100.4 | 1.981 | 12.37 | 7.340 | 15.80 |
| | SD | 23,721 | 541,763 | 2.173 | 13.01 | 6.192 | 8.758 | 1.756 | 2.461 | 1.419 | 4.425 |
| | CV % | 17.9 | 37.0 | 32.2 | 18.9 | 19.6 | 8.7 | 88.6 | 19.9 | 19.3 | 28.0 |
| Cohort 6 | N | 16 | 16 | 16 | 16 | 16 | 16 | 16 | 16 | 16 | 16 |
| (12.0 mg/kg) | Mean | 169,279 | 1,791,460 | 7.192 | 71.43 | 58.56 | 130.0 | 0.9119 | 14.12 | 7.237 | 19.07 |
| | SD | 35,891 | 480,648 | 2.088 | 16.50 | 20.09 | 33.34 | 0.4420 | 4.691 | 2.134 | 6.295 |
| | CV % | 21.2 | 26.8 | 29.0 | 23.1 | 34.3 | 25.6 | 48.5 | 33.2 | 29.5 | 33.0 |

When patients were dosed with the antibody MOR00208 once weekly over an eight week time interval (including an additional loading dose on study day 4), the AUC was calculated over the complete time period (cumulated AUC) and compared to the clinical response. All patients showing a cumulated AUC of at least 14,500 μg*day/mL over eight weeks (corresponding to dose levels 9 and 12 mg/kg only) had a better overall clinical response as shown in Table 4 as well as an significantly increased PFS (see FIG. 6).

1. A method for treatment of chronic lymphocytic leukemia in a patient, said method comprising administering to the patient a pharmaceutical composition comprising an antibody specific for CD19 comprising an HCDR1 region of comprising sequence SYVMH (SEQ ID NO: 1), an HCDR2 region of comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6) in a therapeutically effective amount of 9 mg/kg or more.

2. The method of claim 1 wherein a total exposure to said patient measured by area under the curve (AUC) of 14,500 μg*day/ml or more over at least eight weeks is achieved.

3. The method of claim 1 wherein the pharmaceutical composition is administered at least once weekly over at least eight weeks.

4. The method of claim 1 wherein the pharmaceutical composition is administered intravenously.

5. The method of claim 1 wherein the antibody in a therapeutically effective amount of 15 mg/kg or more.

6. The method of claim 1 wherein the antibody comprises a variable heavy chain of the sequence EVQLVESGGGLVKPGGSLKLSCAASGYTFTSYVMHWVRQAPGKGLEWIGYIN PYNDGTKYNEKFQGRVTISSDKSISTAYMELSSLRSEDTAMYYCARGTYYYGT RVFDYWG QGTLVTVSS (SEQ ID NO: 7) and a variable light chain of the sequence DIVMTQSPATLSLSPGERATLSCRSSKSLQNVNGNTYLYWFQQKPGQSPQLLI YRMSNLNSGVPDRFSGSGSGTEFTLTISSLEPEDFAVYYCMQHLEYPITFGAGT KLEIK (SEQ ID NO: 8).

7. The method according to claim 6, wherein the antibody comprises a heavy chain constant domain of the sequence ASTKGPSVFPLAPSSKSTSGGTAALGCLVKDYFPEPVTVSWNSGALTSGVHTF PAVLQSSGLYSLSSWTVPSSSLGTQTYICNVNHKPSNTKVDKKVEPKSCDKTH TCPPCPAPELLGGPDVFLFPPKPKDTLMISRTPEVTCVVVDVSHEDPEVQFNW YVDGVEVHNAKTKPREEQFNSTFRWSVLTWHQDWLNGKEYKCKVSNKALP APEEKTISKTKGQPREPQVYTLPPSREEMTKNQVSLTCLVKGFYPSDIAVEWES NGQPENNYKTTPPMLDSDGSFFLYSKLTVDKSRWQQGNVFSCSVMHEALHNH YTQKSLSLSPGK (SEQ ID NO: 9).


SEQUENCE LISTING (11 sequences)

SEQ ID NO: 1 - protein, 5 aa, Artificial Sequence
  isolated modified antibody
SYVMH

SEQ ID NO: 2 - protein, 6 aa, Artificial Sequence
  isolated modified antibody
NPYNDG

SEQ ID NO: 3 - protein, 12 aa, Artificial Sequence
  isolated modified antibody
GTYYYGTRVFDY

SEQ ID NO: 4 - protein, 16 aa, Artificial Sequence
  isolated modified antibody
RSSKSLQNVNGNTYLY

SEQ ID NO: 5 - protein, 7 aa, Artificial Sequence
  isolated modified antibody
RMSNLNS

SEQ ID NO: 6 - protein, 9 aa, Artificial Sequence
  isolated modified antibody
MQHLEYPIT

SEQ ID NO: 7 - protein, 121 aa, Artificial Sequence
  isolated modified antibody
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 8 - protein, 112 aa, Artificial Sequence
  isolated modified antibody
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 9 - protein, 330 aa, Artificial Sequence
  isolated modified antibody
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 556 aa, Artificial Sequence [sequence omitted]

SEQ ID NO: 11 - protein, 107 aa, Artificial Sequence [sequence omitted]